(19) United States
(12) Patent Application Publication
(10) Pub. No.: US 2007/0276187 A1
(43) Pub. Date: Nov. 29, 2007

(54) SCANNED BEAM IMAGER AND ENDOSCOPE CONFIGURED FOR SCANNING BEAMS OF SELECTED BEAM SHAPES AND/OR PROVIDING MULTIPLE FIELDS-OF-VIEW

(76) Inventors: Christopher A. Wiklof, Everett, WA (US); Hakan Urey, Istanbul (TR); Selso Luanava, Woodinville, WA (US)

Correspondence Address: Michael G. Pate, Esq.;DORSEY & WHITNEY LLP, Suite 3400, 1420 Fifth Avenue, Seattle, WA, 98101, US

(21) Appl. No.: 11/679,105

(22) Filed: Feb. 26, 2007

Related U.S. Application Data
(60) Provisional application No. 60/777,693, filed on Feb. 27, 2006.

(51) Int. Cl. A61B 1/06; G06K 7/10

(52) U.S. Cl. 600/182; 235/454

(57) ABSTRACT

Scanned beam imagers and endoscopes are disclosed. In one embodiment, a scanned beam imager includes a first light source operable to provide a first beam and a second light source operable to provide a second beam. The scanned beam imager includes a scanner positioned to receive the first and second beams. The scanner is operable to scan the first beam across a FOV as a first scanned beam having a first beam waist distance and the second beam across the FOV as a second scanned beam having a second beam waist distance not equal to the first beam waist distance. A detector is configured to collect reflected light from the FOV. In another embodiment, a scanned beam imager is configured to scan the first and second beams across different FOVs. Such scanned beam imagers may also be incorporated into endoscope tips and bar code scanners.

### CROSS-REFERENCE TO RELATED APPLICATION

This application is based on provisional application No. 60/777,693, filed Feb. 27, 2006.

### TECHNICAL FIELD

This invention relates to scanned beam systems and, more particularly, to scanned beam imagers and endoscopes configured for scanning beams of selected shapes and/or providing multiple fields-of-view (FOVs).

### BACKGROUND

Scanned beam imagers are a promising technology that function by scanning a beam of light over a FOV, collecting the reflected light from the FOV into a small optical sensor, and forming a digital image based on the reflected light. Scanned beam imagers may offer a greater range and depth of field, reduced motion blur, enhanced resolution, extended spectral response, reduced cost, reduced size, lower power consumption, and improved shock and vibration tolerance.

FIG. 1 shows a block diagram of a scanned beam imager 10 according to the prior art. The scanned beam imager 10 includes a light source 12 operable to emit a beam of light 14. A scanner 16 is positioned to receive and scan the beam 14 across a FOV 11 as a scanned beam 18. Instantaneous positions of the scanned beam of light 18 are designated as 18a and 18b. The scanned beam 18 sequentially illuminates spots 20 in the FOV at positions 20a and 20b, respectively. While the scanned beam 18 illuminates the spots, a portion of the illuminating scanned beam 18 is reflected (e.g., specular reflected light and diffuse reflected light also referred to as scattered light), absorbed, refracted, or otherwise affected according to the properties of the object or material at the spots to produce reflected light 22a and 22b. A portion of the reflected light 22a and 22b is received by detector(s) 24, which generates electrical signals corresponding to the amount of light energy received. The electrical signals drive a controller 26 that builds up a digital representation of the FOV and transmits it for further processing, decoding, archiving, printing, display, or other treatment or use via interface 28.

One promising application for a scanned beam imager is in an endoscope. Endoscopes are typically flexible or rigid devices that have an endoscope tip including an imaging unit, such as a digital camera or a scanned beam imager, configured for collecting light and converting the light to an electronic signal. The electronic signal is sent up a flexible tube to a console for display and viewing by a medical professional such as a doctor or nurse.

Scanned beam endoscopes which employ scanned beam imager technology are a fairly recent innovation, and an example of a scanned beam endoscope is disclosed in U.S. patent application Ser. No. 10/873,540 (“'540 application”) entitled SCANNING ENDOSCOPE, hereby incorporated by reference and commonly assigned herewith. FIGS. 2 through 4 show a scanned beam endoscope disclosed in '540 application. As shown in FIG. 2, the scanned beam endoscope 30 includes a control module 32, monitor 34, and optional pump 36, all of which may be mounted on a cart 38, and collectively referred to as console 40. The console 40 communicates with a handpiece 42 through an external cable 44, which is connected to the console 40 via connector 46. The handpiece 42 is operably coupled to the pump 46 and an endoscope tip 54. The handpiece 42 controls the pump 46 in order to selectively pump irrigation fluid through a hose 50 and out of an opening of the endoscope tip 54 in order to lubricate a body cavity that the endoscope tip 54 is disposed within. The endoscope tip 54 includes a distal tip 48 having a scanning module configured to scan a beam across a field-of-view (FOV).

The endoscope tip 54 and distal tip 48 thereof are configured for insertion into a body cavity for imaging internal surfaces thereof. In operation, the distal tip 48 scans a beam of light across a FOV, collects the reflected light from the interior of the body cavity, and sends a signal representative of an image of the internal surfaces to the console 40 for viewing and use by the medical professional.

FIGS. 3 and 4 depict the distal tip 48 and a scanning module 56 of the distal tip 48, respectively, according to the prior art. Referring to FIG. 3, the distal tip 48 includes a housing 58 that encloses and carries the scanning module 56, a plurality of detection optical fibers 60, and an end cap 62 affixed to the end of the housing 58. The detection optical fibers 60 are disposed peripherally about the scanning module 56 within the housing 58. Referring to FIG. 4, the scanning module 56 has a housing 58 that encloses and supports a micro-electro-mechanical (MEMS) scanner 60 and associated components, an illumination optical fiber 62 affixed to the housing 58 by a ferrule 64, and a beam shaping optical element 66. A dome 68 having an interior reflecting surface 74 and an exterior surface 75 is affixed to the end of the housing 58 and may be hermetically sealed thereto in order to protect the sensitive components of the scanning module 56.

In operation, the distal tip 48 is inserted into a body cavity. The illumination optical fiber 62 transmits light 70 to the scanning module 56 and is shaped by the beam shaping optical element 66 to form a selected beam shape. After shaping, a shaped beam 72 is transmitted through an aperture in the center of the MEMS scanner 60, reflected off a reflecting surface 74 of the interior of the dome to the front of the scanner 60, and then reflected off of the scanner 60 as a scanned beam 76 through the dome 68. The dome 68 may further shape the scanned beam 76 to have a beam waist distance 61a selected distance from the end of the dome 68. The scanned beam 76 is scanned across a FOV and reflected off of the interior of a body cavity. At least a portion of the reflected light is collected by the detection optical fibers 60. Accordingly, the reflected light collected by the detection optical fibers 60 may be converted to an electrical signal using optical-electrical converters, such as photodiodes, and the signal representative of an image may be sent to the console 40 for viewing on the monitor 34.

While the scanned beam imager 10 and the scanned beam endoscope 30 are effective imaging devices, the beam waist distance of the scanned beam 18 of the scanned beam imager 10 and the beam waist distance of the scanned beam 76 of the scanned beam endoscope 10 may not be effective for imaging portions of the FOV from different working distances. Moreover, the respective FOVs of the scanned beam imager 10 and the scanned beam endoscope 30 may not be as large as desired.

### SUMMARY

Scanned beam imagers, scanned beam endoscopes, endoscope tips, and methods of use are disclosed. In one aspect, a scanned beam imager includes a first light source operable to provide a first beam and a second light source operable to provide a second beam. The scanned beam imager includes a scanner positioned to receive the first and second beams. The scanner is operable to scan the first beam across a FOV as a first scanned beam having a first beam waist distance and the second beam across the FOV as a second scanned beam having a second beam waist distance not equal to the first beam waist distance. The scanned beam imager further includes a detector configured to detect reflected light from the FOV. The scanned beam imager enables imaging portions of the FOV at different working distances by selecting which of the first and second light sources emits light corresponding to a scanned beam having a beam waist distance suitable for imaging the particular portion of the FOV from the particular working distance.

In another aspect, a scanned beam endoscope includes at least one light source operable to provide light and an endoscope tip. The endoscope tip includes a first illumination optical fiber having an input end coupled to the at least one light source and an output end configured to emit a first beam and at least another illumination optical fiber having an input end coupled to the at least one light source and an output end configured to emit a second beam. The endoscope tip further includes a scanner positioned to receive the first and second beams, the scanner operable to scan the first beam across a FOV as a first scanned beam having a first beam waist distance and the second beam across the FOV as a second scanned beam having a second beam waist distance not equal to the first beam waist distance. The endoscope tip also includes at least one detection optical fiber configured to collect reflected light from the FOV and transmit optical signals characteristic of the FOV. The endoscope includes a converter operable to convert the optical signals to electrical signals and a display coupled to receive the electrical signals from the converter, the display being operable to show an image characteristic of the FOV.

In another aspect, a method of scanning light across a FOV includes positioning a scanned beam imager at a first working distance from a first portion of the FOV and at a second working distance from a second portion of the FOV. A first beam output from the scanned beam imager is scanned across the FOV, the first beam having a first beam waist distance approximately equal to the first working distance. A second beam may be output from the scanned beam imager across the FOV, the second beam having a second beam waist distance approximately equal to the second working distance and not equal to the first beam waist distance. At least a portion of reflected light from the FOV is detected.

In another aspect, a scanned beam imager includes a first light source operable to provide a first beam and a second light source operable to provide a second beam. The scanned beam imager includes a scanner positioned to receive the first and second beams. The scanner is operable to scan the first beam across a first FOV as a first scanned beam and the second beam across a second FOV as a second scanned beam. The scanned beam imager further includes a detector configured to detect reflected light from the first and second FOVs. The scanned beam imager enables providing a larger cumulative FOV.

In another aspect, a scanned beam endoscope includes at least one light source operable to provide light and an endoscope tip. The endoscope tip includes a first illumination optical fiber having an input end coupled to the at least one light source and an output end configured to emit a first beam and at least another illumination optical fiber having an input end coupled to the at least one light source and an output end configured to emit a second beam. The endoscope tip further includes a scanner positioned to receive the first and second beams, the scanner operable to scan the first beam across a first FOV as a first scanned beam and the second beam across a second FOV as a second scanned beam. The endoscope tip also includes at least one detection optical fiber configured to collect reflected light from the first and second FOVs and transmit optical signals characteristic of the first and second FOVs. The endoscope includes a converter operable to convert the optical signals to electrical signals and a display coupled to receive the electrical signals from the converter, the display being operable to show an image characteristic of the first and second FOVs.

In yet another aspect, a method of scanning beams across a plurality of FOVs using a scanned beam imager includes scanning a first beam output from the scanned beam imager across a first FOV. A second beam output from the scanned beam imager may be scanned across a second FOV. At least a portion of the reflected light from the first and second FOVs is detected.

### BRIEF DESCRIPTION OF THE DRAWINGS

FIG. 1 is a block diagram of a scanned beam imager according to the prior art.

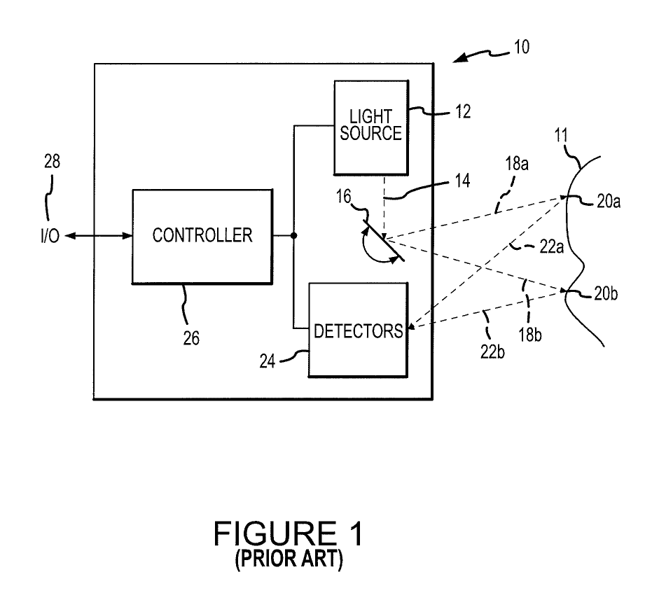

FIG. 2 is schematic drawing of a scanned beam endoscope according to the prior art.

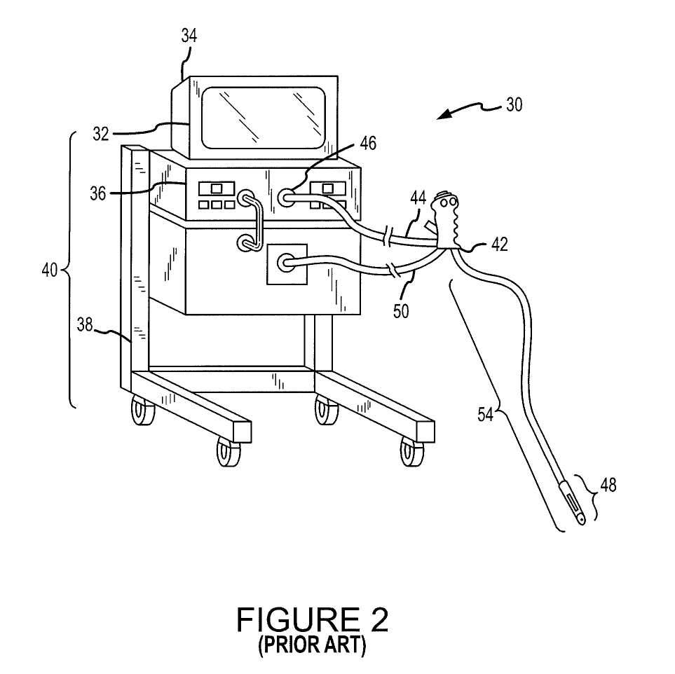

FIG. 3 is a schematic partial isometric view of a distal tip of an endoscope tip shown in FIG. 2 according to the prior art.

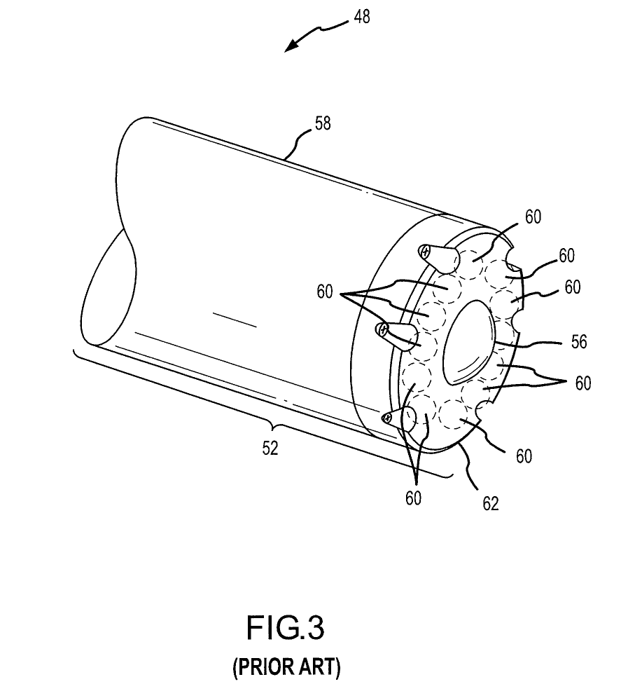

FIG. 4 is a schematic partial side cross-sectional view of the scanning module of FIG. 3 according to the prior art.

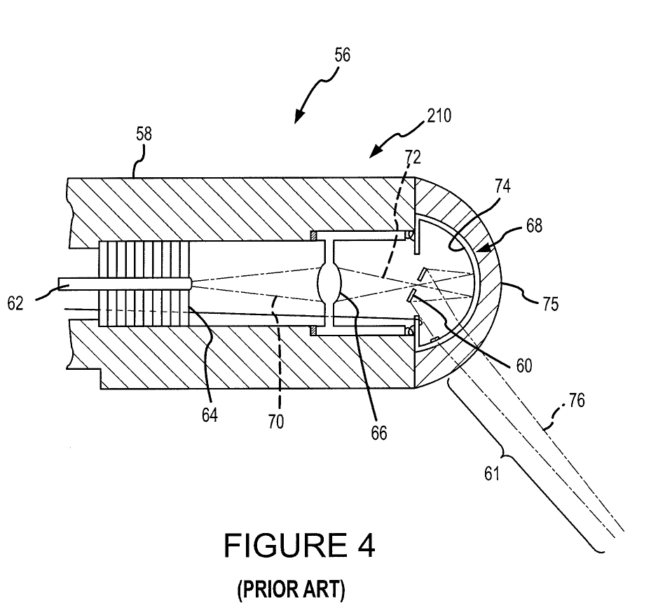

FIG. 5 is a block diagram of one embodiment of a scanned beam imager configured to scan beams having different beam waist distances.

FIG. 6 is a block diagram of another embodiment of a scanned beam imager.

FIG. 7 is a block diagram of yet another embodiment of a scanned beam imager.

FIG. 8 is a block diagram of one embodiment of a scanned beam imager configured to provide a plurality of FOVs.

FIG. 9 is a schematic partial isometric view of a distal tip of an endoscope tip according to one embodiment.

FIG. 10 is a schematic partial side cross-sectional view of the scanning module of FIG. 9 configured to produce and scan beams having various beam waist distances according to one embodiment.

FIG. 11 is a schematic partial side cross-sectional view of a scanning module configured to produce and scan beams across different FOVs according to another embodiment.

FIG. 12 is schematic drawing of a scanned beam endoscope that may utilize any of scanning modules disclosed herein according to one embodiment.

FIG. 13 is a block diagram illustrating the relationship between the various components of the scanned beam endoscope of FIG. 12 according to one embodiment.

### DETAILED DESCRIPTION OF THE EMBODIMENTS

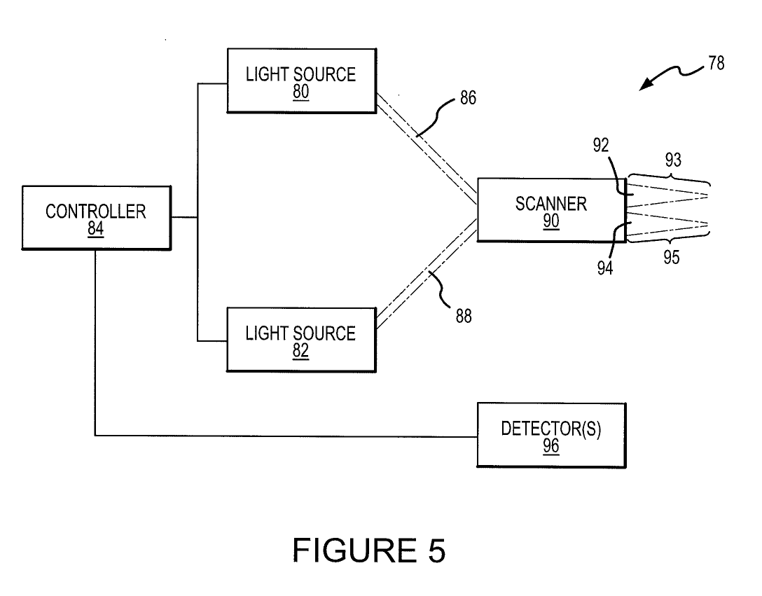

Apparatuses and methods for scanned beam imagers and endoscopes are disclosed. Many specific details of certain embodiments are set forth in the following description and in FIGS. 5 through 13 in order to provide a thorough understanding of such embodiments. One skilled in the art, however, will understand that there may be additional embodiments, or that the disclosed embodiments may be practiced without several of the details described in the following description.

FIG. 5 is a block diagram of one embodiment of a scanned beam imager 78 configured to scan a plurality of scanned beams having different respective beam waist distances. Thus, the scanned beam imager 78 is suitable for imaging a FOV from different working distances. For example, a first scanned beam associated with a first light source and having a first beam waist distance may be suitable for imaging a first potion of the FOV from a first working distance that is approximately equal to the first beam waist distance. A second scanned beam associated with a second light source and having a second beam waist distance may be more suitable for imaging a second portion of the FOV from a second working distance that is approximately equal to the second beam waist distance.

The scanned beam imager 78 includes light sources 80 and 82 operably coupled to a controller 84. The light sources 80 and 82 are operable to emit corresponding beams 86 and 88 having different respective beam shapes, such as beams having different divergence or convergence angles. The controller 84 is configured to cause the light sources 80 and 82 to selectively emit beams 86 and 88 responsive to instructions from the controller 84. In various embodiments, each of the light sources 80 and 82 may be a laser, a light emitting diode, a laser diode, and diode-pumped solid state (DPSS) laser, an optical fiber which may have a focusing or collimating element attached thereto optically coupled to any of the aforementioned devices, or another suitable light source. In one embodiment, the light sources 80 and 82 may be combined into a single light source. The scanned beam imager 78 further includes a scanner 90 positioned to receive the beams 86 and 88, and operable to scan the beams 86 and 88 received from the light sources 80 and 82 across a FOV shown as scanned beams 92 and 94. One or more detectors 96 are provided to receive and detect at least a portion of the light reflected from the FOV (e.g., specular reflected light and diffuse reflected light also referred to as scattered light). In various embodiments, the detector 96 may be a PIN photodiode, avalanche photodiode (APD), photomultiplier tube, one or more optical fibers optical coupled to any of the aforementioned devices, or another suitable detector.

According to various embodiments, the scanner 90 may be a 2D MEMS scanner, such as a bulk micro-machined MEMS scanner assembly, a surface micro-machined device, another type of conventional MEMS scanner, or a subsequently developed MEMS scanner. The scanner 90 may be configured to scan one or more beams of light at high speed and in a pattern that covers an entire FOV or a selected portion of a 2D FOV within a frame period. As known in the art, such MEMS scanners may be driven magnetically, electrostatically, capacitively, or combinations thereof. For example, the horizontal scan motion may be driven electrostatically and the vertical scan motion may be driven magnetically. Electrostatic driving may include electrostatic plates, comb drives or the like. Alternatively, both the horizontal and vertical scan may be driven magnetically or capacitively.

In operation, one of the light sources 80 and 82 selectively emits a beam. For example, the light source 80 emits the beam 86 such as a convergent beam, as shown in FIG. 5, having a first beam waist distance. The beam 86 is received by and scanned by the scanner 90 across the FOV shown as the scanned beam 92. The scanned beam 92 may have a beam waist distance 93 that is suitable for imaging the FOV or a portion of the FOV from a first working distance. The detector 96 receives the reflected light from the FOV. The detector 96 generates electrical signals corresponding to the amount of reflected light energy received. The electrical signals are sent to the controller 84 that builds up a digital representation of the FOV and may transmit it for further processing. The light source 82 may then emit the beam 88 such as a convergent beam, as shown in FIG. 5, having a second beam waist distance that is not equal to the first beam waist distance of the beam 86. The beam 88 is received by and scanned by the scanner 90 across the FOV shown as the scanned beam 94. The scanned beam 94 may have a second beam waist distance that is different from that of the scanned beam 92 associated with the light source 80. This second beam waist distance 95 of the scanned beam 94 may be suitable for imaging the FOV or a portion of the FOV from a second working distance. For example, the first beam waist distance may be relatively shorter and, thus, more suitable for imaging the FOV or a portion of the FOV from a closer working distance while the second beam waist distance may be relatively longer and, thus, suitable for imaging the FOV from a greater working distance. Accordingly, the scanned beam imager 78 may have more than the two light sources 80 and 82 to collectively provide a scanned beam imager 78 with a very large depth of field.

In other embodiments, the beams 86 and 88 may be divergent beams and the scanner 90 may be configured with optical power to shape the beams 86 and 88 to be convergent beams having different respective beam waist distances. In such an embodiment, the light sources 80 and 82 are positioned different distances from the scanner 90 in order to collimate the beams 86 and 88 to different extents.

In one embodiment of the scanned beam imager 78, the light sources 80 and 82 may be optical fibers each having lenses attached to the ends configured so that the beams 86 and 88 each are focused to have different beam waist distances. Each of the optical fibers may be positioned to directly emit the beams 86 and 88 onto the scanner 90, which scans the beams 86 and 88 as scanned beams 92 and 94 having different respective beam waist distances.

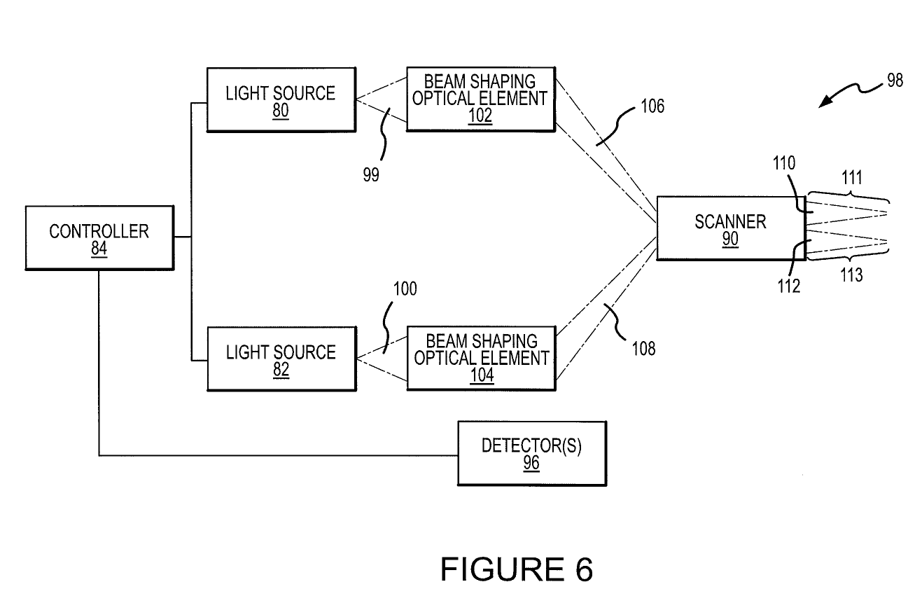

FIG. 6 is a block diagram of another embodiment of a scanned beam imager 98 configured to scan at least two scanned beams having different beam waist distances. The scanned beam imager 98 has many of the same components that are included in the scanned beam imager 78 of FIG. 5. Therefore, in the interest of brevity, the components of the two scanned beam imagers 78 and 98 that correspond to each other have been provided with the same or similar reference numerals, and an explanation of their structure and operation will not be repeated. As shown in FIG. 6, the light sources 80 and 82 may be configured to emit corresponding beams 99 and 100. The beams 99 and 100 are received by corresponding beam shaping optical elements 102 and 104 that are configured to shape the beams 99 and 100 to have selected beam shapes (e.g., selected convergence or divergence angle) shown as beams 106 and 108. The shape of the beam 106 is different from the shape of the beam 108. For example, the beam 106 may be shaped to have a different beam waist distance than that of the beam waist distance of beam 108. In various embodiments, the beam shaping optical elements 99 and 100 may be lenses, doublets, clipping apertures, reflectors, diffractive elements, refractive elements, combinations thereof, or other suitable optical elements.

As with the scanned beam imager 78, the beams 106 and 108 are received by and scanned by the scanner 90 across the FOV shown as scanned beam 110 having a beam waist distance 111 and scanned beam 112 having a beam waist distance 113. Reflected light from the FOV is received by the detector 96 and an image of the FOV may be generated. As with the scanned beam imager 78, the scanned beam imager 98 is also configured to scan at least two beams having different respective beam waist distances to provide a larger depth of field.

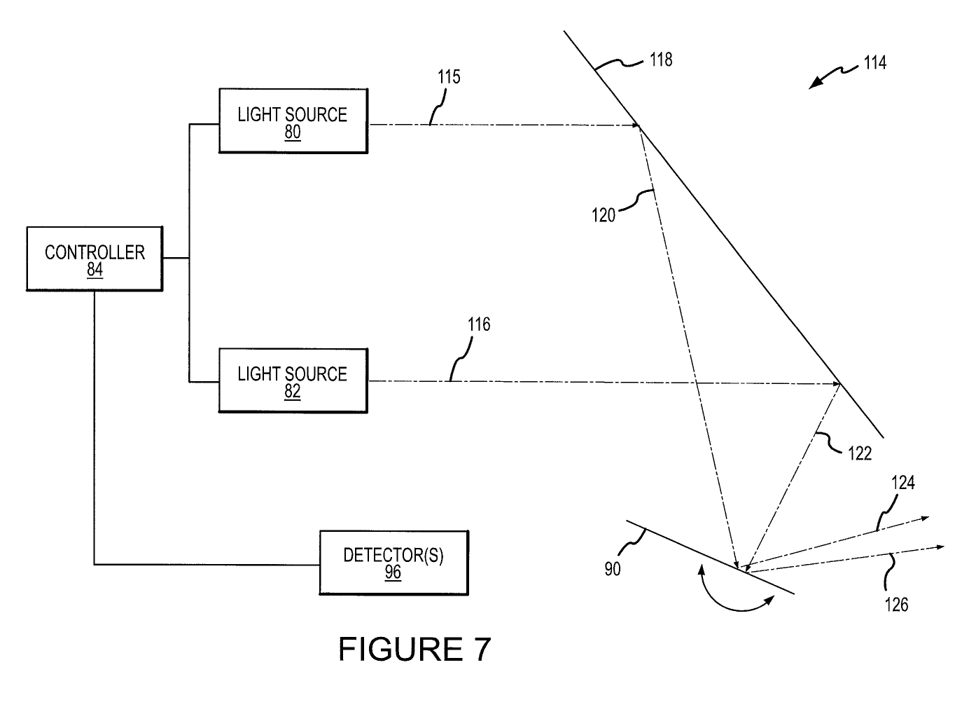

FIG. 7 is a block diagram of yet another embodiment of a scanned beam imager 114 configured to scan beams of different shapes. The scanned beam imager 114 has many of the same components that are included in the scanned beam imager 78 of FIG. 5. Therefore, in the interest of brevity, the components of the scanned beam imagers 78 and 114 that correspond to each other have been provided with the same or similar reference numerals, and an explanation of their structure and operation will not be repeated. As with the scanned beam imager 78, the light sources 80 and 82 selectively emit corresponding beams 115 and 116 shown in FIG. 7 with only their respective central rays. A reflective surface 118 is positioned to receive and redirect the beams 115 and 116 to the scanner 90 shown as redirected beams 120 and 122. In some embodiments, the reflective surface 118 may be a plane mirror, a curved mirror (e.g., a spherical mirror), or another suitable optical element that may have optical power to shape the beams 115 and 116. In one embodiment, the reflective surface 118 is curved to have optical power and the light sources 80 and 82 are positioned different distances from the reflective surface 118 so that the reflective surface 118 may shape the beams 120 and 122 reflected thereby to have different convergence or divergence angles. The scanner 90 is positioned to receive the redirected beams 120 and 122 and scan them across the FOV shown as scanned beams 124 and 126 having different respective beam shapes (e.g., different respective beam waist distances) and the corresponding reflected light is received by the detector 96.

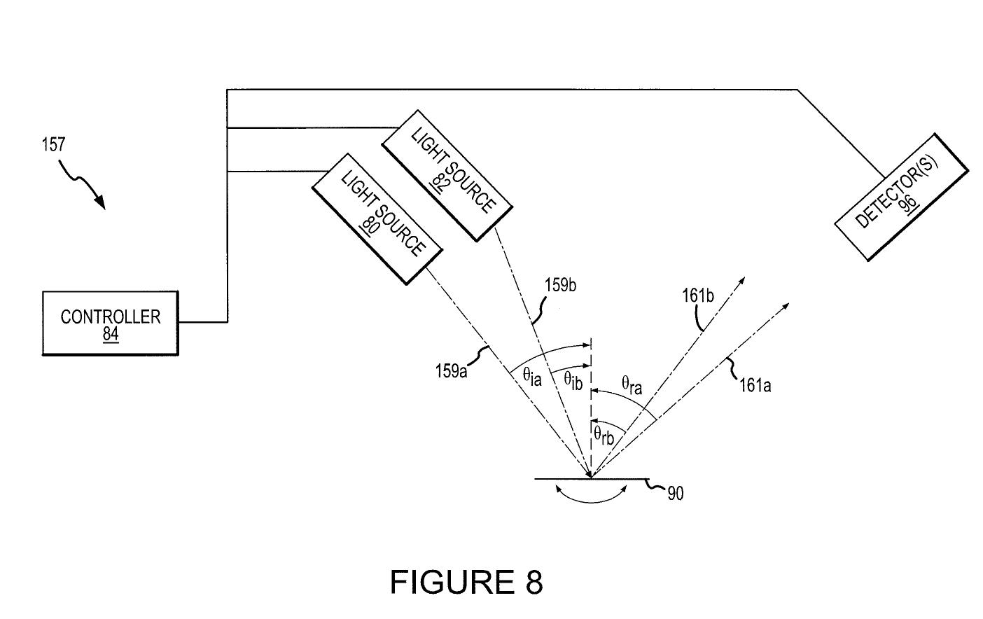

FIG. 8 is a block diagram of an embodiment of a scanned beam imager 157 that is configured to scan beams associated with different light sources across different FOVs. Accordingly, the scanned beam imager 157 may provide a large cumulative FOV. Although the scanned beam imager 157 may be practiced using the embodiments of the scanned beam imagers 78, 98, and 114 of FIGS. 5 through 7 wherein each light source is associated with a scanned beam having a different beam waist distance, more typically, each of the scanned beams may have the same or similar beam waist distance but each scanned beam is scanned across a different FOV to provide a large cumulative FOV.

Referring to FIG. 8, each of the light sources 80 and 82 emits a corresponding beam 159a and 159b incident on the scanner 90 at angle θᵢₐ-θ_{ib} relative to the vertical. In a typical embodiment, the beams 159a and 159b may have the same or similar beam shape. The beams 159a and 159b are reflected from the scanner 90, for a given scanner position, at different angles. The beam 159a is reflected from the scanner 90, shown as scanned beam 161a, at an angle θᵣₐ relative to the vertical and the beam 159b is reflected from the scanner 90, shown as scanned beam 161b, at an angle θ_{rb} relative to the vertical. Accordingly, the scanner 90 may scan the scanned beams 161a and 161b across respective FOVs. The scanned beam 161a is associated with a first FOV and the scanned beam 161b is associated with a second FOV.

In one embodiment, the respective FOVs associated with each of the light sources 80 and 82 and scanned beams 161a and 161b may overlap. In another embodiment, the respective FOVs associated with each of the light sources 80 and 82 and scanned beams 161a and 161b may define a larger substantially contiguous FOV. In yet another embodiment, the respective FOVs associated with each of the light sources 80 and 82 and scanned beams 161a and 161b may be offset from each other.

In one embodiment, a particular FOV may be selected by controlling, using the controller 84, which particular one of the light sources 80 and 82 outputs light. In another embodiment, the light sources 80 and 82 may emit the beams 159a-159b simultaneously or substantially simultaneously and the scanned beams 161a and 161b may be scanned at substantially the same time to provide a larger FOV. In such an embodiment, the individual FOVs may be joined together during image processing. In one embodiment, the particular FOV associated with a respective scanned beam 161a and 161b may be isolated during or after detection of reflected light from the FOV by wavelength, time or frequency multiplexing the optical signals received by the detector 96.

As with the scanned beam imagers 78, 98, and 114 embodiments of FIGS. 5 through 7, the scanned beam imager 157 may also employ beam shaping optical elements to shape the light output from the light sources 80 and 82 and/or a reflective surface such as a plane mirror or curved mirror to redirect the beams 159a and 159b and optionally further shape them. In the interest of brevity, the explanation of such details is not discussed again.

One application of the scanned beam imagers 78, 98, 114, and 157 embodiments of FIGS. 5 through 8 is in a scanned beam endoscope. Any of the aforementioned scanned beam imagers may be incorporated into a distal tip of an endoscope tip for use in a scanned beam endoscope.

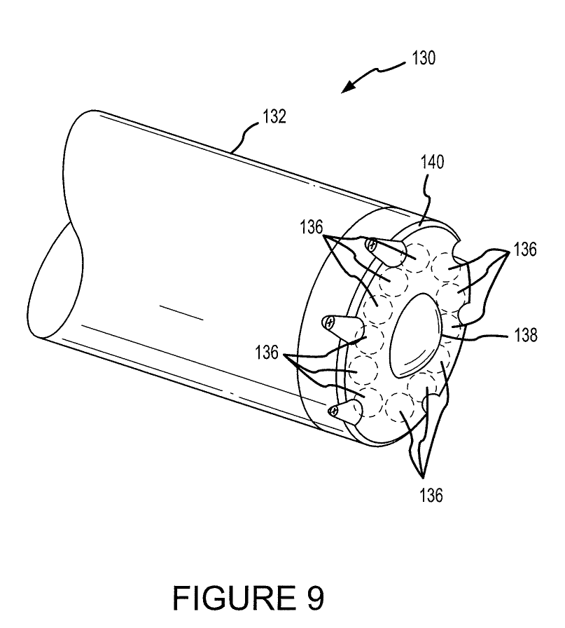

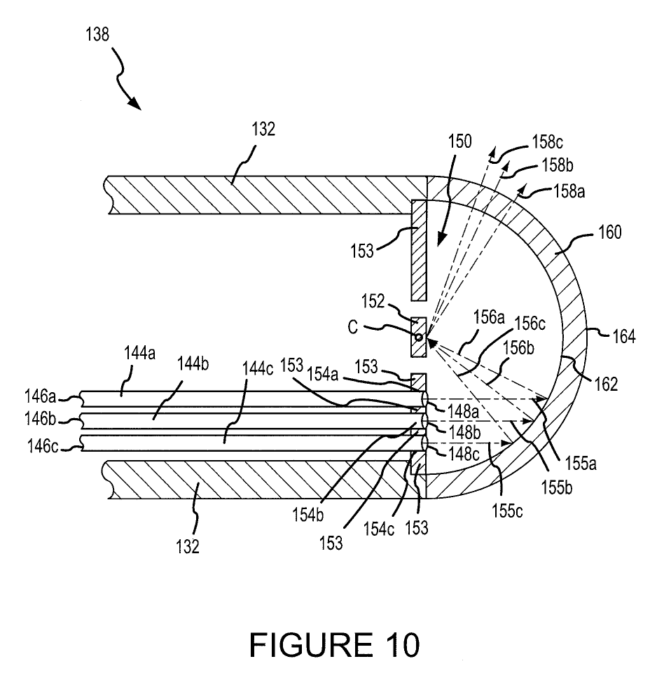

FIGS. 9 and 10 show a distal tip 130 of an endoscope tip and a scanning module 138 of the distal tip 130, respectively, according to one embodiment. The scanning module 138 is adapted to function in a manner similar to the scanned beam imager 114 of FIG. 7. The scanning module 138 of the distal tip 130 is configured to scan beams having selected beam waist distances suitable for imaging a FOV or portions of the FOV from a particular working distance. The distal tip 130 includes a housing 132 that encloses and carries the scanning module 138 and an end cap 140 affixed to the end of the housing 132. The distal tip 130 also includes a plurality of detection optical fibers 136 that may be positioned behind the end cap 140 and may be disposed about the scanning module 138. The end cap 140 is configured to allow at least a portion of light reflected from the FOV to be transmitted through it for collection by the detection optical fibers 136.

Referring to FIG. 10, the scanning module 138 includes a housing 140 that encloses a scanner 152 and a plurality of illumination optical fibers 144a-144c having corresponding input ends 146a-146c and output ends 148a-148c. Although three illumination optical fibers 144a-144c are shown, more than or less than three illumination optical fibers may be used depending upon the particular scanning module design. The input ends 146a-146c may be coupled to one or more light sources (not shown). The scanning module 138 includes a scanner 150 mounted to the interior of the housing 132. The scanner 150 include a scan plate 152 attached to a scan frame 153 in a conventional manner to enable rotation about one or two axes to scan light across a 1D or 2D FOV.

A plurality of laterally distributed vias 154a-154c extend through the scan frame 153 and receive a corresponding one of the illumination optical fibers 144a-144c. The illumination optical fibers 144a-144c may be secured within the vias 154a-154c using a suitable adhesive, such as an epoxy. A dome 160 having an exterior surface 164 and a partially reflective interior surface 162 is attached to the housing 162. In one embodiment, the partially reflective interior surface 162 may be configured to focus or collimate light reflected thereby. In some embodiments, the dome 160 may be configured to reflect and transmit light of a selected polarization direction. Such a dome 160 is disclosed in the aforementioned '540 application. In some embodiments, the dome 160 may be configured to provide optical power for shaping light that passes through it. In other embodiments, the dome 160 may act as a window and a fixed mirror may be disposed between the interior surface 162 of the dome 160 and the scanner 150 to provide the same or similar functionality.

In operation, beams 155a-155c are selectively output from corresponding illumination optical fibers 144a-144c (only the central ray of the beams 155a-155c is shown in FIG. 10 for clarity). Each of the beams 155a-155c may have a different beam waist distance measured axially from the output ends 148a-148c. For example, each of the illumination optical fibers 144a-144c may include a lens or other suitable optical element attached to its end. Such optical fibers are commercially available from Corning Inc. and the lenses may be configured to provide the beams 155a-155c with selected beam waist distances. The beams 155a-155c are reflected and redirected to the scan plate 152 shown as redirected beams 156a-156c (again, only the central ray of the redirected beams 156a-156c is shown in FIG. 10 for clarity). The redirected beams 156a-156c are scanned across a FOV shown as scanned beams 158a-158c having different respective beam waist distances from the exterior surface 164 of the dome 160 (again, only the central ray of the scanned beams 158a-158c is shown in FIG. 10 for clarity). As previously discussed, the dome 160 may further shape the scanned beams 158a-158c. The scanned beams 158a-158c are reflected from the FOV and the reflected light is collected by the detection optical fibers 136 (shown in FIG. 9). The optical signals collected by the detection optical fibers 136 are representative of characteristics of the FOV and may be further processed to define an image.

In another embodiment, the illumination optical fibers 144a-144c may be disposed radially about center C of the scan plate 152. In such an embodiment, the scanned beams 158a-158c reflected from the scanner 150 do not exhibit a significant amount of divergence relative to each other.

In one embodiment, the scanning module 138 is operable so that the each of the illumination optical fibers 144a-144c may selectively emit the beams 154a-154c. In such an embodiment, the distal tip 130 may be positioned within a body cavity so that the exterior surface 164 of the scanning module 138 is positioned a first working distance from a first portion of the FOV and a second working distance from a second portion of the FOV. One of the illumination optical fibers 144a-144c associated with a corresponding one of the scanned beams 158a-158c having a first beam waist distance approximately equal to the first working distance may selectively output a corresponding beam 154a-154c to image the FOV and the first portion of the FOV with a high resolution. Thereafter, one of the illumination optical fibers 144a-144c associated with a corresponding one of the scanned beams 158a-158c having a second beam waist distance approximately equal to the second working distance may selectively output a corresponding beam 154a-154c which is scanned across the FOV to image the FOV and the second portion of the FOV with a high resolution. The process of selectively scanning one of the scanned beams 158a-158c associated with a corresponding illumination optical fiber 144a-144c may be repeated, as desired, so that one of the scanned beams 158a-158c having a beam waist distance suitable for the working distance from the FOV or a particular portion the FOV is used. Accordingly, the distal tip 130 provides a very large effective depth of field.

In another embodiment, the scanning module 138 is operable so that the each of the illumination optical fibers 144a-144c may emit the beams 154a-154c simultaneous or substantially simultaneously and the optical signals associated with each of the illumination optical fibers 144a-144c may be isolated during or after detection of reflected light from the FOV by wavelength, time, or frequency multiplexing the optical signals received by the detection optical fibers 136 (shown in FIG. 9).

Although the scanning module 138 of the distal tip 130 was described above employing a scanned beam imager device very similar to the scanned beam imager 114 of FIG. 7, the scanned beam imagers 78 and 98 of FIGS. 5 and 6 may be adapted for use in a distal tip of an endoscope tip. For example, the dome 160 may be configured as a transparent window and used merely to seal and protect the components of the scanning module 138. Instead of redirecting the beams 154a-154c off of the interior surface 162 of the dome 160 or another fixed mirror, the illumination optical fibers 144a-144c may direct the beams 154a-154c output therefrom directly onto the scan plate 152 of the scanner 150. Other variations and adaptations of the disclosed scanned beam imagers may be employed to enable selectively scanning beams having different beam waist distances from the exterior surface 164 of the dome 160.

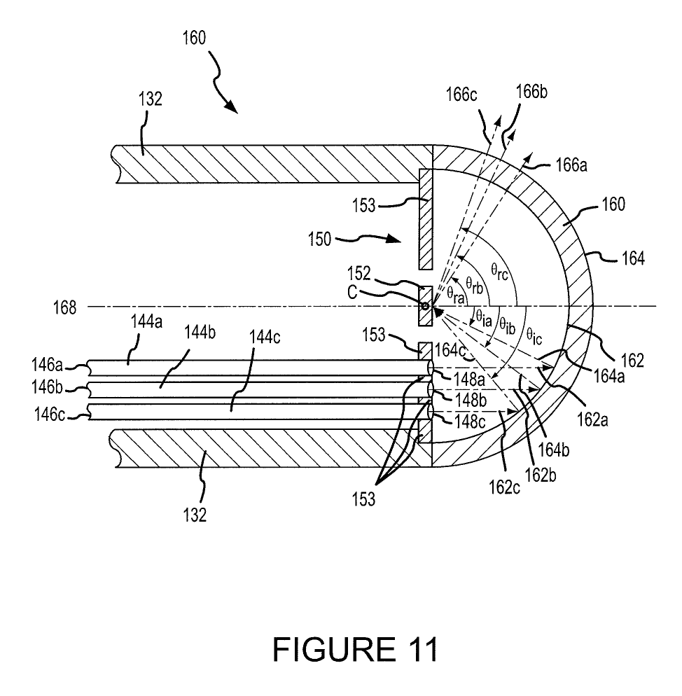

FIG. 11 shows a scanning module 160 for use in a distal tip of an endoscope tip configured to scan beams across a plurality of FOVs according to one embodiment. Thus, the scanning module 160 and distal tip is an adaptation of the scanned beam imager 157 shown in FIG. 8. This operating mode may be used with any of the aforementioned scanning module embodiments in which the illumination optical fibers 144a-144c are positioned to provide scanned beams that are divergent from one another. The scanning module 160 has many of the same components that are included in the scanning module 138 of FIG. 10. Therefore, in the interest of brevity, the components of the two scanning modules 138 and 160 that correspond to each other have been provided with the same or similar reference numerals, and an explanation of their structure and operation will not be repeated. In FIG. 11, only the central rays of the various beams are illustrated for clarity.

The illumination optical fibers 144a-144c output corresponding beams 162a-162c. The beams 162a-162c are reflected and redirected to the scan plate 152 by the interior surface 162 of the dome 160 at an angle θᵢₐ-θ_{ic} relative to a centerline 168 of the scan plate 152 shown as redirected beams 164a-164c. The redirected beams 164a-164c are scanned across a plurality of FOVs shown as scanned beams 166a-166c. Again, for clarity, only the central ray of the scanned beams 166a-166c is shown in FIG. 11. For a given scan angle of the scan plate 152, the scanned beams 166a-166c are reflected from the scan plate 152 at an angle θᵣₐ-θ_{rc} relative to the centerline 168 of the scan plate 152. Accordingly, the scanned beams 166a-166c may be scanned across respective FOVs.

In one embodiment, the respective FOVs associated with each of the illumination optical fibers 144a-144c and scanned beams 166a-166c may overlap. In another embodiment, the respective FOVs associated with each of the illumination optical fibers 144a-144c and scanned beams 166a-166c may define a larger substantially contiguous FOV. In yet another embodiment, the respective FOVs associated with each of the illumination optical fibers 144a-144c and scanned beams 166a-166c may be offset from each other.

In one embodiment, a particular FOV may be selected by controlling which particular one of the illumination optical fibers 144a-144c outputs light. In another embodiment, all of the illumination optical fibers 144a-144c may emit the beams 162a-162c simultaneously or substantially simultaneously and the scanned beams 166a-166c may be scanned at substantially the same time to provide a larger FOV. In such an embodiment, the individual FOVs may be joined together during image processing. In one embodiment, the particular FOV associated with a respective scanned beam 166a-166c may be isolated during or after detection of reflected light from the FOV by wavelength, time, or frequency multiplexing the optical signals received by the detection optical fibers 136 (shown in FIG. 8).

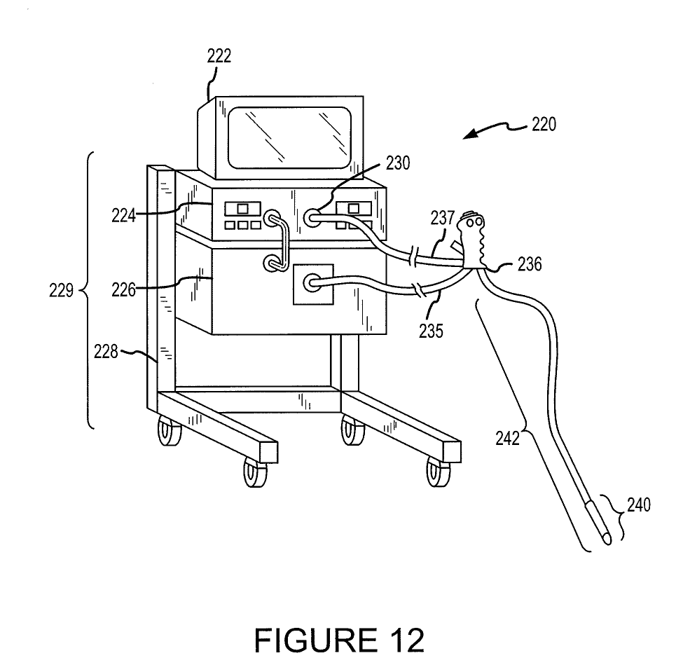

FIG. 12 shows a schematic drawing of a scanned beam endoscope 220 according to one embodiment that may utilize any of the aforementioned embodiments of distal tips and associated scanning modules. The scanned beam endoscope 220 includes a control module 224, monitor 222, and optional pump 226, all of which may be mounted on a cart 228, and collectively referred to as console 229. The console 229 communicates with a handpiece 236 through an external cable 237, which is connected to the console 229 via connector 230. The handpiece 236 may be operably coupled to the pump 226 and an endoscope tip 242. The handpiece 236 controls the pump 226 in order to selectively pump irrigation fluid through a hose 235 and out of an opening of the endoscope tip 242. The endoscope tip 242 includes a distal tip 240, which may be any of the aforementioned distal tips. The endoscope tip 242 encloses components of the distal tip 240, such as optical fibers and electrical wiring, and, optionally, other components such as an irrigation channel, a working channel, and a steering mechanism.

In operation, the distal tip 240 is placed within a body cavity. Responsive to user input via the handpiece 236, the distal tip 240 scans light over the FOV. Reflected light from an interior surface of the body cavity is collected by the distal tip 240. A signal representative of an image of the internal surfaces is sent from the distal tip 240 of the endoscope 220 to the console 229 for viewing on the monitor 222 and diagnosis by the medical professional.

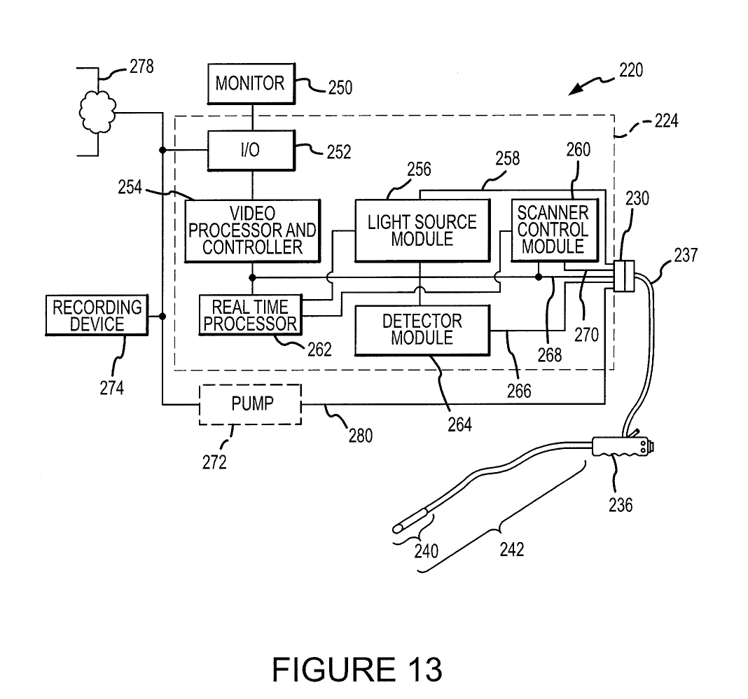

FIG. 13 is a block diagram illustrating the relationships between various components of the endoscope 220 in more detail. The control module 224 contains a number of logical and/or physical elements that cooperate to produce an image on the monitor 222. The control module 224 includes a video processor and controller 254 that receives and is responsive to control inputs by the user via the handpiece 236. The video processor and controller 254 may also include image processing functions. The user control inputs are sent to the video processor and controller 254 via a control line 268.

The video processor and controller 254 also controls the operation of the other components within the control module 224. The control module 224 further includes a real time processor 262 coupled to the video processor and controller 254, which may, for example, be embodied as a PCI board mounted on the video processor and controller 254. The real time processor 262 is coupled to a light source module 256, a scanner control module 260, a detector module 264, and the video processor and controller 254. The scanner control module 260 is operable to control the scanner of the scanned beam endoscope 240 and the detector module 264 is configured for detecting light reflected from the FOV.

The light source module 256, which may be housed separately, includes one or more light sources that provides the light energy used for beam scanning by the distal tip 240. Suitable light sources for producing polarized and/or non-polarized light include light emitting diodes, laser diodes, and diode-pumped solid state (DPSS) lasers. Such light sources may also be operable to emit light over a range of wavelengths. In one embodiment, each of the illumination optical fibers of the distal tip 240 may be coupled to a corresponding light source. In another embodiment, a single light source may be coupled to all of the illumination optical fibers of the distal tip 240 and in a manner to enable selectively coupling light to a particular one of the illumination optical fibers.

Responsive to user inputs via the handpiece 236, a control signal is sent to the video processor and controller 254 via the control line 268. The video processor and controller 254 transmits instructions to the real time processor 262. Responsive to instructions from the real time processor 262, light energy is output from the light source module 256 to the scanned beam endoscope 240 via an optical fiber 258. The optical fiber 258, which is optically coupled to the external cable 237 via the connector 230, transmits the light to the external cable 237. The light travels through the handpiece 236 to the scanned beam endoscope 240 and is ultimately scanned across the FOV. Light reflected from the FOV is collected at the distal tip 240 by the detection optical fibers (not shown) and a representative signal is transmitted to the controller module 224.

In some embodiments, the representative signal transmitted to the control module 224 is an optical signal. Thus, a return signal line 266 may be an optical fiber or an optical fiber bundle that is coupled to the detector module 264 and transmit the representative optical signal to the detector module 264. At the detector module 264, the optical signals corresponding to the FOV characteristics are converted into electrical signals and returned to the real time processor 262 for real time processing and parsing to the video processor and controller 254. Electrical signals representative of the optical signals may be amplified and optionally digitized by the detector module 264 prior to transmission to real time processor 262. In an alternative embodiment, analog signals may be passed to the real time processor 262 and analog-to-digital conversion performed there. It is also contemplated that the detector module 264 and the real time processor 262 may be combined into a single physical element.

In other embodiments, light representative of the FOV may be converted into electrical signals at the distal tip 240 or the endoscope tip 242 by one or more photo-detectors such as PIN photodiodes, avalanche photodiodes (APDs), or photomultiplier tubes. In such an embodiment, the return line 266 may be electrical wires and the detector module 264 may be omitted.

The video processor and controller 254 has an interface 252 that may include several separate input/output lines. A video output may be coupled to the monitor 222 for displaying the image. A recording device 274 may also be coupled to the interface 252 to capture video information recording a procedure. Additionally, in some embodiments, the endoscope system 220 may be connected to a network or the Internet 278 for remote expert input, remote viewing, archiving, library retrieval, or the like. In another embodiment, the video processor and controller 254 may optionally combine data received via the interface 252 with image data and the monitor 222 with information derived from a plurality of sources including the distal tip 240.

In another embodiment, in addition to or as an alternative to the monitor 222, the image may be output to one or more remote devices such as, for example, a head mounted display. In such an embodiment, context information such as viewing perspective may be combined with FOV and/or other information in the video processor and controller 254 to create context-sensitive information display.

From the foregoing it will be appreciated that, although specific embodiments of the invention have been described herein for purposes of illustration, various modifications may be made without deviating from the spirit and scope of the invention. For example, various other embodiments for a scanned beam imager may be used to scan beams having different respective beam waist distances and/or scan beams across different respective FOVs. Additionally, such scanned beam imagers may be incorporated into a variety of apparatuses such as scanned beam endoscopes and bar code scanners. Accordingly, the invention is not limited except as by the appended claims.

What is claimed is:

1. A scanned beam imager for use in a scanned beam endoscope, comprising:
a first light source operable to provide a first beam;
a second light source operable to provide a second beam;
a scanner positioned to receive the first and second beams, the scanner operable to scan the first beam across a field-of-view (FOV) as a first scanned beam having a first beam waist distance and the second beam across the FOV as a second scanned beam having a second beam waist distance not equal to the first beam waist distance; and
a detector configured to detect reflected light from the FOV.

2. The scanned beam imager of claim 1 wherein:
the first light source is operable to emit the first beam with a first beam shape; and
the second light source is operable to emit the second beam with a second beam shape that is different than the first beam shape.

3. The scanned beam imager of claim 2 wherein the first beam shape comprises a first convergence or divergence angle and the second beam shape comprises a second convergence or divergence angle.

4. The scanned beam imager of claim 1 wherein the first and second light sources are positioned to direct the first and second beams directly onto the scanner.

5. The scanned beam imager of claim 1, further comprising:
a first beam shaping optical element positioned to receive the first beam and configured to shape the first beam to a first beam shape; and
a second beam shaping optical element positioned to receive the second beam and configured to shape the second beam to a second beam shape that is different than the first beam shape.

6. The scanned beam imager of claim 5 wherein each of the first and second beam shaping optical elements comprises at least one lens, a clipping aperture, a reflector, a diffractive element, a refractive element, or combinations thereof.

7. The scanned beam imager of claim 1, further comprising:
a reflective surface positioned to receive the first and second beams and oriented to direct the first and second beams to the scanner.

8. The scanned beam imager of claim 7 wherein:
the reflective surface has optical power;
the first light source is spaced apart from the reflective surface a first distance; and
the second light source is spaced apart from the reflective surface a second distance not equal to the first distance.

9. The scanned beam imager of claim 1 wherein each of the first and second light sources comprises a laser, a light emitting diode, a laser diode, or an optical fiber light source.

10. The scanned beam imager of claim 1 wherein the detector comprises a PIN photodiode, avalanche photodiode (APD), or a photomultiplier tube.

11. The scanned beam imager of claim 1 wherein the scanner is configured to have optical power.

12. The scanned beam imager of claim 1 wherein the scanner comprises a MEMS scanner.

13. The scanned beam imager of claim 1, further comprising a controller configured to cause the first and second light sources to selectively emit the first and second beams.

14. A method of scanning light across a field-of-view (FOV), comprising:
positioning a scanned beam imager at a first working distance from a first portion of the FOV and at a second working distance from a second portion of the FOV;
scanning a first beam output from the scanned beam imager across the FOV, the first beam having a first beam waist distance approximately equal to the first working distance;
scanning a second beam output from the scanned beam imager across the FOV, the second beam having a second beam waist distance approximately equal to the second working distance and not equal to the first beam waist distance; and
detecting at least a portion of reflected light from the FOV.

15. The method of claim 14 wherein the acts of scanning a first beam output from the scanned beam imager across the FOV, the first beam having a first beam waist distance approximately equal to the first working distance and scanning a second beam output from the scanned beam imager across the FOV, the second beam having a second beam waist distance approximately equal to the second working distance and not equal to the first beam waist distance comprises selectively scanning the first and second beams.

16. The method of claim 14 wherein the acts of scanning a first beam output from the scanned beam imager across the FOV, the first beam having a first beam waist distance approximately equal to the first working distance and scanning a second beam output from the scanned beam imager across the FOV, the second beam having a second beam waist distance approximately equal to the second working distance and not equal to the first beam waist distance comprises scanning the first and second beams substantially simultaneously.

17. The method of claim 14, further comprising:
isolating optical signals associated with the first scanned beam and the second scanned beam reflected from the FOV.

18. The method of claim 14 wherein:
the act of scanning a first beam output from the scanned beam imager across the FOV, the first beam having a first beam waist distance approximately equal to the first working distance comprises directing the first beam from a first location to a scanner;
the act of scanning a second beam output from the scanned beam imager across the FOV, the second beam having a second beam waist distance approximately equal to the second working distance and not equal to the first beam waist distance comprises directing the second beam from a second location to the scanner.

19. The method of claim 14 wherein:
the act of scanning a first beam output from the scanned beam imager across the FOV, the first beam having a first beam waist distance approximately equal to the first working distance comprises:
emitting the first beam from a first location; and
reflecting the first beam to a scanner;
the act of scanning a second beam output from the scanned beam imager across the FOV, the second beam having a second beam waist distance approximately equal to the second working distance and not equal to the first beam waist distance comprises:
emitting the second beam from a second location; and
reflecting the second beam to the scanner.

20. The method of claim 14 wherein:
the act of scanning a first beam output from the scanned beam imager across the FOV, the first beam having a first beam waist distance approximately equal to the first working distance comprises:
emitting the first beam from a first location spaced apart from a reflecting surface a first distance; and
reflecting the first beam to a scanner;
the act of scanning a second beam output from the scanned beam imager across the FOV, the second beam having a second beam waist distance approximately equal to the second working distance and not equal to the first beam waist distance comprises:
emitting the second beam from a second location spaced apart from a reflecting surface a second distance that is not equal to the first distance; and
reflecting the second beam to the scanner.

21. The method of claim 14 wherein:
the act of scanning a first beam output from the scanned beam imager across the FOV, the first beam having a first beam waist distance approximately equal to the first working distance comprises:
emitting first light from a first location; and
shaping the first light to a first beam shape;
the act of scanning a second beam output from the scanned beam imager across the FOV, the second beam having a second beam waist distance approximately equal to the second working distance and not equal to the first beam waist distance comprises:
emitting second light from a second location; and
shaping the second light to a second beam shape that is different than that of the first beam shape.

22. The method of claim 14 wherein the scanned beam imager is included in a scanned beam endoscope.

23. A scanned beam imager, comprising:
a first light source operable to provide a first beam;
a second light source operable to provide a second beam;
a scanner positioned to receive the first and second beams, the scanner operable to scan the first beam across a first field-of-view (FOV) as a first scanned beam and the second beam across a second FOV as a second scanned beam; and
a detector configured to detect reflected light from the first and second FOVs.

24. The scanned beam imager of claim 23 wherein the first and second FOVs overlap.

25. The scanned beam imager of claim 23 wherein the first and second FOVs do not substantially overlap.

26. The scanned beam imager of claim 23 wherein the scanner is positioned to receive and, for a given scan position of the scanner, reflect the first and second scanned beams at different relative angles.

27. The scanned beam imager of claim 23 wherein the first and second beams are directed onto the scanner at different respective angles of incidence.

28. The scanned beam imager of claim 23 wherein the first and second light sources are positioned to direct the first and second beams directly onto the scanner.

29. The scanned beam imager of claim 23, further comprising:
a first beam shaping optical element positioned to receive the first beam and configured to shape the first beam to a first beam shape; and
a second beam shaping optical element positioned to receive the second beam and configured to shape the second beam to a second beam shape that is different from the first beam shape.

30. The scanned beam imager of claim 23 wherein each of the first and second beam shaping optical elements comprises at least one lens, a clipping aperture, a reflector, a diffractive element, a refractive element, or combinations thereof.

31. The scanned beam imager of claim 23, further comprising:
a reflective surface positioned to receive the first and second beams and oriented to direct the first and second beams to the scanner.

32. The scanned beam imager of claim 23 wherein each of the first and second light sources comprises a laser, a light emitting diode, a laser diode, or an optical fiber light source.

33. The scanned beam imager of claim 23 wherein the detector comprises a PIN photodiode, avalanche photo diode (APD), or a photomultiplier tube.

34. The scanned beam imager of claim 23 wherein the scanner is configured to have optical power.

35. The scanned beam imager of claim 23 wherein the scanner comprises a MEMS scanner.

36. A method of scanning beams across a plurality of fields-of-view (FOVs) using a scanned beam imager, comprising:
scanning a first beam output from the scanned beam imager across a first FOV;
scanning a second beam output from the scanned beam imager across a second FOV; and
detecting at least a portion of reflected light from the first and second FOVs.

37. The method of claim 36 wherein the acts of scanning a first beam across a second FOV output from the scanned beam imager across a second FOV and scanning a second beam output from the scanned beam imager across a second FOV comprises substantially simultaneously scanning the first beam across the first FOV and the second beam across the second FOV.

38. The method of claim 36, further comprising selectively displaying an image associated with reflected light from one of the first FOV and the second FOV.

39. The method of claim 36 wherein:
the act of scanning a first beam output from the scanned beam imager across a first FOV comprises reflecting the first beam from a scanner at a first angle; and
the act of scanning a second beam output from the scanned beam imager across a second FOV comprises reflecting the second beam from the scanner at a second angle.

40. The method of claim 36 wherein:
the act of scanning a first beam output from the scanned beam imager across a first FOV comprises:
emitting the first beam from a first location;
redirecting the first beam to a scanner; and
scanning the redirected first beam across the first FOV; and
the act of scanning a second beam output from the scanned beam imager across a second FOV comprises:
emitting the second beam from a second location;
redirecting the second beam to the scanner; and
scanning the redirected second beam across the second FOV.

41. The method of claim 36 wherein the acts of scanning a first beam output from the scanned beam imager across a first FOV and scanning a second beam output from the scanned beam imager across a second FOV comprises scanning the first and second beams using a MEMS scanner.

42. The method of claim 36 wherein the scanned beam imager is included in a scanned beam endoscope.

43. The method of claim 36 wherein the first and second FOVs overlap.

44. The method of claim 36 wherein the first and second FOVs are substantially contiguous.

45. A scanned beam endoscope, comprising:
at least one light source;
an endoscope tip, comprising:
a first illumination optical fiber having an input end coupled to the at least one light source and an output end configured to emit a first beam;
at least another illumination optical fiber having an input end coupled to the at least one light source and an output end configured to emit a second beam;
a scanner positioned to receive the first and second beams, the scanner operable to scan the first beam across a field-of-view (FOV) as a first scanned beam having a first beam waist distance and the second beam across the FOV as a second scanned beam having a second beam waist distance not equal to the first beam waist distance;
at least one detection optical fiber configured to collect reflected light from the FOV and transmit optical signals characteristic of the FOV;
a converter operable to convert the optical signals to electrical signals; and
a display coupled to receive the electrical signals from the converter, the display being operable to show an image characteristic of the FOV.

46. The scanned beam endoscope of claim 45, further comprising: a controller coupled to the at least one light source, the controller operable to selectively couple the light from the at least one light source to the first and at least another illumination optical fibers.

47. The scanned beam endoscope of claim 45 wherein:
the first illumination optical fiber is configured to emit the first beam with a first beam shape; and
the at least another illumination optical fiber is configured to emit the second beam with a second beam shape that is different than the first beam shape.

48. The scanned beam endoscope of claim 47 wherein the first beam shape is shaped to have the first beam waist distance and the second beam shape is shaped to have the second beam waist distance.

49. The scanned beam endoscope of claim 45 wherein the first and at least another illumination optical fibers are positioned to direct the first and second beams directly onto the scanner.

50. The scanned beam endoscope of claim 45, further comprising:
a first beam shaping optical element positioned to receive the first beam and configured to shape the first beam to a first beam shape; and
a second beam shaping optical element positioned to receive the second beam and configured to shape the second beam to a second beam shape that is different than the first beam shape.

51. The scanned beam endoscope of claim 50 wherein each of the first and second beam shaping optical elements comprises at least one lens, a clipping aperture, a reflector, a diffractive element, a refractive element, or combinations thereof.

52. The scanned beam endoscope of claim 45, further comprising:
a reflective surface positioned to receive the first and second beams and oriented to direct the first and second beams to the scanner.

53. The scanned beam endoscope of claim 52 wherein:
the reflective surface has optical power;
the output end of the first illumination optical fiber is spaced apart from the reflective surface a first distance; and
the output end of the at least another illumination optical fiber is spaced apart from the reflective surface a second distance not equal to the first distance.

54. The scanned beam endoscope of claim 52 wherein the reflective surface comprises an interior surface of a dome of the endoscope tip.

55. The scanned beam endoscope of claim 45 wherein the at least one light source comprises a laser, a light emitting diode, or a laser diode.

56. The scanned beam endoscope of claim 45 wherein the scanner is configured to have optical power.

57. The scanned beam endoscope of claim 45 wherein the scanner comprises a MEMS scanner.

58. The scanned beam endoscope of claim 45 wherein the at least one detection optical fiber comprises a plurality of detection optical fibers positioned about the scanner.

59. A scanned beam endoscope, comprising:
at least one light source operable to provide light;
an endoscope tip, comprising:
a first illumination optical fiber having an input end coupled to the at least one light source and an output end configured to emit a first beam;
at least another illumination optical fiber having an input end coupled to the at least one light source and an output end configured to emit a second beam;
a scanner positioned to receive the first and second beams, the scanner operable to scan the first beam across a first field-of-view (FOV) as a first scanned beam and the second beam across a second FOV as a second scanned beam;
at least one detection optical fiber configured to collect reflected light from the first and second FOVs and transmit optical signals characteristic of the first and second FOVs;
a converter operable to convert the optical signals to electrical signals; and
a display coupled to receive the electrical signals from the converter, the display being operable to show an image characteristic of the first and second FOVs.

60. The scanned beam endoscope of claim 59 wherein the first and second FOVs overlap.

61. The scanned beam endoscope of claim 59 wherein the first and second FOVs do not substantially overlap.

62. The scanned beam endoscope of claim 59 wherein the scanner is positioned to receive and, for a given scan position of the scanner, reflect the first and second scanned beams at different relative angles.

63. The scanned beam endoscope of claim 59 wherein the first and second beams are directed onto the scanner at different respective angles of incidence.

64. The scanned beam endoscope of claim 59, further comprising: a controller coupled to the at least one light source, the controller operable to selectively couple the light from the at least one light source to the first and at least another illumination optical fibers.

65. The scanned beam endoscope of claim 59 wherein:
the first illumination optical fiber is configured to emit the first beam with a first beam shape; and
the at least another illumination optical fiber is configured to emit the second beam with a second beam shape that is different than the first beam shape.

66. The scanned beam endoscope of claim 65 wherein the first beam shape is shaped to have the first beam waist distance and the second beam shape is shaped to have the second beam waist distance.

67. The scanned beam endoscope of claim 59 wherein the first and at least another illumination optical fibers are positioned to direct the first and second beams directly onto the scanner.

68. The scanned beam endoscope of claim 59, further comprising:
a first beam shaping optical element positioned to receive the first beam and configured to shape the first beam to a first beam shape; and
a second beam shaping optical element positioned to receive the second beam and configured to shape the second beam to a second beam shape that is different than the first beam shape.

69. The scanned beam endoscope of claim 68 wherein each of the first and second beam shaping optical elements comprises at least one lens, a clipping aperture, a reflector, a diffractive element, a refractive element, or combinations thereof.

70. The scanned beam endoscope of claim 59, further comprising:
a reflective surface positioned to receive the first and second beams and oriented to direct the first and second beams to the scanner.

71. The scanned beam endoscope of claim 70 wherein the reflective surface comprises an interior surface of a dome of the endoscope tip.

72. The scanned beam endoscope of claim 70 wherein:
the reflective surface has optical power;
the output end of the first illumination optical fiber is spaced apart from the reflective surface a first distance; and
the output end of the at least another illumination optical fiber is spaced apart from the reflective surface a second distance not equal to the first distance.

73. The scanned beam endoscope of claim 59 wherein each of the first and second light sources comprises a laser, a light emitting diode, or a laser diode.

74. The scanned beam endoscope of claim 59 wherein the scanner is configured to have optical power.

75. The scanned beam endoscope of claim 59 wherein the scanner comprises a MEMS scanner.

76. The scanned beam endoscope of claim 59 wherein the at least one detection optical fiber comprises a plurality of detection optical fibers positioned about the scanner.

77. An endoscope tip, comprising:
a first illumination optical fiber having an input end coupled to the at least one light source and an output end configured to emit a first beam;
at least another illumination optical fiber having an input end coupled to the at least one light source and an output end configured to emit a second beam;
a scanner positioned to receive the first and second beams, the scanner operable to scan the first beam across a field-of-view (FOV) as a first scanned beam having a first beam waist distance and the second beam across the FOV as a second scanned beam having a second beam waist distance not equal to the first beam waist distance; and
at least one detection optical fiber configured to collect reflected light from the FOV and transmit optical signals characteristic of the FOV.

78. An endoscope tip, comprising:
a first illumination optical fiber having an input end coupled to the at least one light source and an output end configured to emit a first beam;
at least another illumination optical fiber having an input end coupled to the at least one light source and an output end configured to emit a second beam;
a scanner positioned to receive the first and second beams, the scanner operable to scan the first beam across a first field-of-view (FOV) as a first scanned beam and the second beam across a second FOV as a second scanned beam; and
at least one detection optical fiber configured to collect reflected light from the first and second FOVs and transmit optical signals characteristic of the first and second FOVs.
